(19) United States
(12) Patent Application Publication
(10) Pub. No.: US 2009/0155188 A1
(43) Pub. Date: Jun. 18, 2009

(54) MOUTH ENVIRONMENT WITH ORAL COMPOSITION

(76) Inventors: F. Richard Austin, Layton, UT (US); Blaine D. Austin, Layton, UT (US)

Correspondence Address: JAMES SONNTAG;JAMES SONNTAG, PATENT ATTORNEY, P.O. BOX 9194, SALT LAKE CITY, UT, 84109, US

(21) Appl. No.: 12/248,295

(22) Filed: Oct. 9, 2008

Related U.S. Application Data
(60) Provisional application No. 60/978,752, filed on Oct. 9, 2007.

(51) Int. Cl. A61K 8/22; A61K 8/66

(52) U.S. Cl. 424/48; 424/53; 424/50

(57) ABSTRACT

A solid oral composition for providing a mouthwash efficacious effect comprising a non-hygroscopic base, carbamide peroxide, a bacterial immobilizing and sulfur sequestration agent, and an optional antioxidant enzyme.

### RELATED APPLICATIONS

This application claims priority from U.S. Provisional Patent Application 60/978,752, filed 9 Oct. 2007, and U.S. patent application Ser. No. 11/377,223, filed Mar. 14, 2006, which are hereby incorporated by reference.

### BACKGROUND

Compositions for whitening teeth are have been made. Examples of such compositions are disclosed in U.S. Pat. No. 6,149,211, Ser. No. 09/028,330, issued 21 Nov. 2000, to Losee et al., which is hereby incorporated by reference.

### SUMMARY

The present disclosure involves a composition and method for manufacture for a solid orally administered formulation. The formulation is water-free and the ingredients are a base of a non-hygroscopic sweetener, carbamide peroxide, a bacterial immobilizing agent and sulfur sequestration agent that includes chlorine dioxide, and an antioxidant. The base may be any suitable non-hygroscopic sweetener, but may, for example, be mannitol, or a hydrogenation product of isomaltulose, such as the disaccharide, Isomalt™, or mixtures thereof.

The composition of the present disclosure has the effect of improving the mouth environment by, among other things, raising the pH and reducing bacterial growth (which reduces cavity formation and gingivitis, and improves the healing of wounds). The compositions of present disclosure are useful in the prevention and reduction of bad breath, plaque and related gum diseases, and effective in killing, and/or altering the bacterial metabolism, and/or for a period of time suppressing the growth to microorganisms which cause topically-treatable infections and diseases of the oral cavity, such as plaque, gingivitis, periodontal disease, and breath malodor. The composition also contains an antioxidant to inhibit cellular changes in the user that may otherwise occur due to the oxidants in the composition.

The present disclosure provides the same effect as a liquid mouthwash, but with a solid composition taken orally in the form of a tablet or the like.

The composition of the present disclosure does not degrade and has a long shelf-life. But, as the product is orally administered and is dissolved by the saliva while being chewed or held in the mouth, it reacts with the saliva to produce an effect that improves the mouth environment without producing an unpleasant taste. It has been found that any urea that is formed in this process of dissolution in the mouth is insufficient to impart a bad taste to the product.

### BRIEF DESCRIPTION OF THE DRAWINGS

FIG. 1 is a flow-sheet of a method of manufacturing an exemplary composition of the present disclosure.

FIG. 2 is a flow-sheet of another method of manufacturing an exemplary composition of the present disclosure.

### DETAILED DESCRIPTION

The compositions of the present disclosure comprise the ingredients, a non-hygroscopic base or sweetener composition, carbamide peroxide, chlorine dioxide, a bacterial immobilizing agent, and. The composition may also contain an antioxidant enzyme.

### Non-Hygroscopic Sweetener Base

For purposes of this disclosure the base composition is the portion of the composition other than the carbamide peroxide, the bacterial immobilization agent, the chlorine dioxide, and any antioxidant enzyme. The major portion (>50 wt. %) of base composition comprises a non-hygroscopic sweetener base as the sole or principal sweetener. Any non-hygroscopic sweetener is suitable. This may be one of or a mixture of mannitol, and a hydrogenation product of isomaltulose. Other sweeteners are suitable, if they have the essentially the same or better non-hydroscopic properties as mannitol.

By “non-hygroscopic” is meant a composition lacking a hygroscopic property, i.e., the tendency of the substance to hydrate by absorbing water, usually from the ambient humidity. The base material should be non-hygroscopic at ambient temperatures and humidities usually encountered during handling and storage. The composition may comprise materials that are to some degree hygroscopic, but they must be present in small enough amounts, such that the essentially non-hygroscopic nature of the entire base composition is retained. Basically, sufficient non-hygroscopic properties enable a product to contain negligible water to react with the carbamide peroxide. This permits the composition to have an adequate shelf life without developing a bad taste from hydration products of the carbamide peroxide.

The base also comprises other materials used conventionally to form the appropriate paste, gum, tablet or candy. The base is essentially water-free and non-hygroscopic. By “water-free” is meant that it contains negligible water that is free to react with the carbamide peroxide. Components of the composition may contain waters of hydration, but they must be bound sufficiently to the base composition or be present in such a small amount to provide negligible reactive water.

A suitable composition for the sweetener of the base composition is mannitol, a catalytically hydrogenation product of isomaltulose (6-0-.alpha.-D-Glucopyranosyl-D-fructose), or mixtures thereof. The catalytic hydrogenation of isomaltulose (a derivative of sucrose) produces two stereoisomers, 6-0-.alpha.-D-Glucopyranosyl-D-sorbitol (1,6-GPS) and 1-0-.alpha.-D-Glucopyranosyl-D-mannitol (1,1-GPM). (These are also known as “-D-glucopyranosyl-1,6-D-sorbitol and “-D-glucopyranosyl-1,1-D-mannitol, respectively.) The hydrogenation reaction is an equimolar mixture of the two isomeric disaccharide polyols, 1,6-GPS and 1,1-GPM. Isomalt is sold under the trademark Palatinit™ by Palatinit Sussungsmittel GmbH, Mannheim, Germany. Isomalt is non-hygroscopic, particularly at ambient temperatures and humidities. At 25 degrees Celsius, Isomalt absorbs very little water. At temperatures as high as 60 degrees Celsius and 80 degrees Celsius the relative humidity must be 75% and 65% respectively for significant water absorption. A detailed discussion of Isomalt, its manufacture and various properties is disclosed in Isomalt, by Peter Strater and William E. Irwin in Alternative Sweeteners, edited by Lyn O'Brien Nabors and Robert C. Gellardi, Marcel Dekker, Inc., 1992, and ISOMALT, 5th edition, published in 1996 by Platinit Sussungsmittel GmbH, Gottlieb-Daimler-Stra.beta.e, D-68165, Mannheim, Germany. The hydrogenation reaction of isomaltulose is disclosed in U.S. Pat. No. 4,117,173 to Schiweck et al. The base composition may comprise other suitable ingredients that are used in candy and tablet compositions, such as other non-hygroscopic sweeteners, flavors, sweeteners, anti-oxidants, stabilizers, colorants, and the like.

### Carbamide Peroxide

An active ingredient in the present composition is carbamide peroxide, CON₂H₄.H₂O₂. It is the addition compound of hydrogen peroxide and urea and is also known as urea peroxide, or urea hydrogen peroxide.

The composition contains carbamide peroxide in an effective amount, i.e., an amount to be efficacious to improve the mouth environment in combination with the other active ingredients, when the composition is taken orally and dissolved in the mouth or chewed to dissolve and release the carbamide peroxide and other active ingredients.

An efficacious amount includes amounts of carbamide peroxide below about 3 weight percent, and below those amounts below where the carbamide peroxide provides a tooth-whitening effect. An aspect of the present disclosure includes compositions comprising carbamide peroxide and a non-hygroscopic base where the carbamide peroxide is in an amount insufficient to provide an effective or satisfactory tooth-whitening effect, but sufficient to provide an efficacious amount for bacterial immobilization.

### Bacterial Immobilizing and Sulfur Sequestration Agent

The present compositions include a bacterial immobilizing and sulfur sequestration agent (BISSA). The BISSA comprises chlorine dioxide, either alone or optionally one or more of any other compound that is effective as a bacterial immobilizing agent and/or a sulfur sequestration agent. By “bacterial immobilizing” is meant prevention, suppression, or inhibition of bacterial growth. Thus, for the bacterial immobilizing agent bactericidal and bacteriostatic compositions or mixtures thereof are suitable. These include antibacterial agents that are often commonly employed in oral compositions and that are compatible with the other components in the non-hygroscopic environment of the composition and are activated in the mouth environment.

Sulfur sequestration agents include any suitable compound that will remove volatile sulfur compounds from the mouth environment. These include oxidant materials, may of which also have bacterial immobilizing properties. These sulfur compounds are a contributor to bad breath, and their removal is desirable.

Specific examples of components in addition to chlorine dioxide include, but are not limited to, and biguanides, such as chlorhexidine digluconate, and mixtures thereof. Examples may also possibly include quaternary ammonium antibacterial compounds and water-soluble sources of certain metal ions such as zinc, copper, silver and stannous (e.g., zinc, copper and stannous chloride, and silver nitrate).

### Antioxidant Enzyme

In order to increase the safety of the present composition, the present composition optionally comprises an antioxidant. Any antioxidant that is used in food products, any product taken or ingested orally, or oral treatments is suitable. Suitable antioxidants include any of various antioxidant enzymes. The antioxidant is added to increase the safety of components of the composition that have sufficient oxidant concentration to potentially cause cellular changes from exposure to the composition.

A suitable antioxidant enzyme is Coenzyme Q₁₀. Coenzyme Q₁₀ (also known as ubiquinone, ubidecarenone, coenzyme Q and abbreviated at times to CoQ10, CoQ, Q10 or Q) is a benzoquinone, where the “Q” and the “10” in the name refer to the quinone chemical group and the 10 isoprenyl chemical subunits, respectively. Coenzyme Q₁₀ is known for its antioxidant properties and is used in dietary supplements.

### Method of Manufacture

The composition is made by any suitable process where carbamide peroxide is incorporated into the solid base material such that the carbamide peroxide is protected or shielded from humidity in the atmosphere, and no water or no hygroscopic materials that would absorb water are introduced that would result in water being introduced into the composition during processing or storage in an amount that would produce a taste-detectable amount of urea over several days or weeks.

Carbamide peroxide when reacted with water decomposes into urea. In the compositions of the present disclosure, the carbamide peroxide is isolated from water while in the product, so that it cannot react with water. This is accomplished by not adding any water during manufacture of the composition, and using materials that are as free from water as practical. In addition, the components of the composition are preferably non-hygroscopic and do not attract water from the atmosphere. In addition, the composition must have a physical and chemical nature that protects the carbamide peroxide from humidity in the air. It has been found, that some oral compositions with certain non-hygroscopic sweeteners are unsatisfactory and bad-tasting. While not completely understood, it is believed that the unsatisfactory taste in these certain products may be due to an unsatisfactory taste of the sweetener itself, and/or from urea produced because the base material does not adequately isolate the carbamide peroxide from atmospheric moisture. Thus, the non-hygroscopic nature of the sweetener-base is the most important, but not the only factor. However, it has been found that when a non-hygroscopic sweetener, such as, for example, a hydrogenation product of isomaltulose and/or mannitol is used as the sweetener base, the urea carbamide is stabilized and protected and will not degrade over several weeks of storage.

Certain prior-art gum, tablet and candy compositions contain water, or often contain substances that attract water from the humidity in the air. In normal gum, tablet and candy compositions, this water is no problem. But if carbamide peroxide is present, that water is available to react with and degrade the carbamide peroxide to urea, which is bad tasting even in very small concentrations.

In the method of the present disclosure, materials that are essentially free of water are used, or water is expelled from the composition. Because of the inherent properties of the base of the composition, there is no readsorbtion or attraction of water from the humidity in the air. Accordingly, the carbamide peroxide is protected or shielded from water and the decomposition reaction does not take place sufficiently to produce an unpleasant taste. Accordingly, it has been found that the compositions of the present composition can be stored for several weeks with no detectable deterioration in taste and with an insignificant loss of the active ingredient.

When the composition is placed within the mouth and is allowed to dissolve or is chewed, the composition slowly dissolves in the mouth, which provides an effective amount of the carbamide peroxide and bacterial immobilizing and sulfur sequestration agent to improve the environment of the mouth. Preferably, a gum, candy or tablet is formed so as to dissolve the carbamide peroxide and bacterial immobilizing agent slowly over about a 20 minute period. A consistent daily use of the tablets will then obtain a useful effect for the consumer.

One method for manufacturing the composition comprises first heating the base material to a temperature sufficient to drive off any water in the composition. For Isomalt, this is a temperature of about 150 degrees Celsius or above. The base material is then cooled to a temperature at which the carbamide peroxide and the bacterial immobilizing and sulfur sequestration agent can be incorporated and mixed into the base material. As the temperature approaches near is 120 degrees Celsius, the carbamide peroxide disassociates. Accordingly, the preferred mixing temperature is 118 degrees Celsius or below. When Isomalt cools to near 110 degrees Celsius it becomes difficult to mix the carbamide peroxide in an Isomalt base. Accordingly the preferred temperature for mixing in Isomalt is 114 degrees Celsius or above. In summary, for mixing carbamide peroxide in Isomalt, the mixing temperature is preferably between 114 degrees Celsius and 118 degrees Celsius, more preferably about 116 degrees Celsius.

After incorporating the carbamide peroxide and the BISSA, the material is then allowed to solidify. An antioxidant enzyme is also optionally added. The solid base can be formed into hard candy shapes by any suitable method, or crushed into powder and then hard pressed in to tablets, or otherwise processed by conventional methods into a solid form that can be orally administered. Basically, any process that permits the heating to expel the water and the mixing temperature of the carbamide peroxide is suitable. The other components of the composition, i.e., the sweeteners, stabilizers, flavors, etc. are added at any suitable time, as dictated by their properties.

Alternately, a composition may be produced by compressing into the tablets a powdered mixture of the components of the present composition.

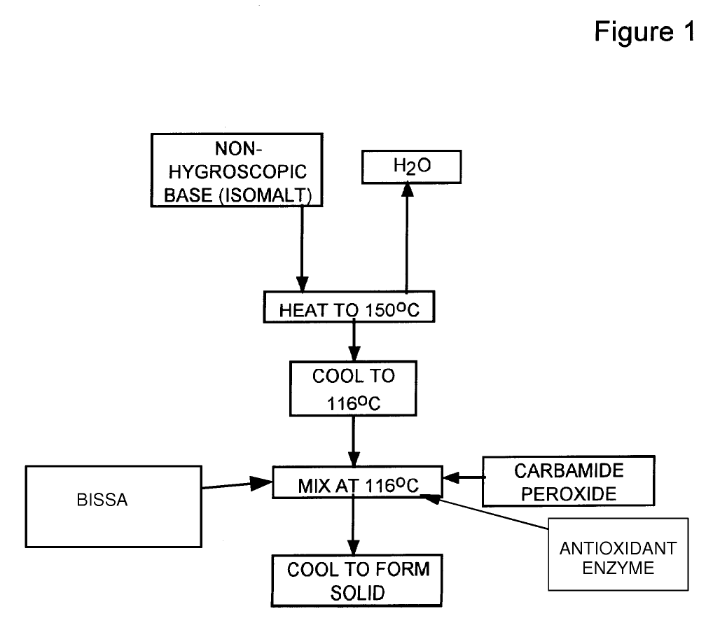

Referring to FIG. 1, which is a flow sheet of a method for manufacture, a non-hygroscopic base, as defined herein, is provided and heated to 150 degrees Celsius to drive of the water in the base. The base is cooled to 116 degrees Celsius, the temperature at which carbamide peroxide is added and mixed. The material is then allowed to cool to form a solid material.

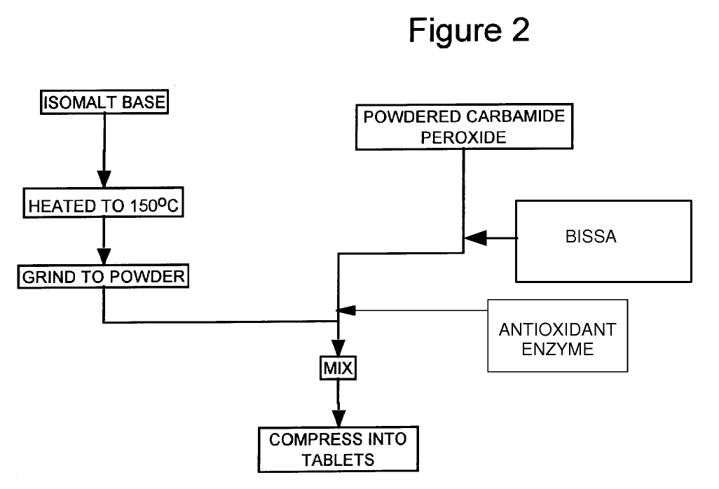

FIG. 2 is a flow sheet of an alternate method for manufacture of the composition. A non-hygroscopic base is provided optionally heated to 150 degrees Celsius. to drive off moisture. The base is then ground and mixed with powdered carbamide peroxide and BISSA. The mixture is then compressed into tablets by conventional techniques.

What is claimed is:

1. A non-hygroscopic solid oral composition comprising:
carbamide peroxide in an amount effective to dissolve and produce a efficacious improvement in mouth environment when the composition is dissolved in mouth;
bacterial immobilizing and sulfur sequestration agent comprising chlorine dioxide;
a solid, water-free, non-hygroscopic base;
with water in the composition at sufficiently low amount and the composition being sufficiently non-hygroscopic to avoid a detectable taste of urea from the reaction of carbamide peroxide with water.

2. A non-hygroscopic solid oral composition as in claim 1 also comprising an antioxidant.

3. A non-hygroscopic solid oral composition as in claim 1 wherein the antioxidant is an enzyme

4. A non-hygroscopic solid oral composition as in claim 3 wherein the antioxidant enzyme is Coenzyme Q₁₀.

5. A non-hygroscopic solid oral composition as in claim 1 wherein the bacterial immobilizing and sulfur sequestration agent also comprises chlorhexidine digluconate.

6. A non-hygroscopic solid oral composition as in claim 1 wherein the bacterial immobilizing and sulfur sequestration agent also comprises a one of or a mixture of biguanide, a quaternary ammonium antibacterial compound, or water-soluble source of zinc, copper, silver or stannous.

7. A non-hygroscopic solid oral composition as in claim 1 wherein the non-hygroscopic base comprises a hydrogenation product of isomaltulose.

8. A non-hygroscopic solid oral composition as in claim 1 wherein the non-hygroscopic base comprises mannitol.

9. A non-hygroscopic solid oral composition as in claim 1 wherein the composition is in the form of a tablet.

10. A non-hygroscopic solid oral composition as in claim 1 wherein the composition is in the form of a candy.

11. A non-hygroscopic solid oral composition as in claim 1 wherein the composition is in the form of a gum.

12. A method for manufacturing a solid oral composition comprising
compounding into a mixture an efficacious amount of carbamide peroxide, a bacterial immobilizing and sulfur sequestration agent comprising chlorine dioxide, and a non-hygroscopic base in a water-free environment,
forming the mixture into a solid shape.

13. A method for manufacturing a solid oral composition as in claim 10 wherein an antioxidant is also compounded into the mixture.

14. A method for manufacturing a solid oral composition as in claim 10 wherein the antioxidant is an enzyme and the enzyme is Coenzyme Q₁₀.

15. A non-hygroscopic solid oral composition comprising:
carbamide peroxide in an amount effective to dissolve and produce a efficacious improvement in mouth environment when the composition is dissolved in mouth;
bacterial immobilizing and sulfur sequestration agent comprising chlorine dioxide;
a solid, water-free, non-hygroscopic base including a hydrogenation product of isomaltulose;
an antioxidant enzyme including Coenzyme Q₁₀;
with water in the composition at sufficiently low amount and the composition being sufficiently non-hygroscopic to avoid a detectable taste of urea from the reaction of carbamide peroxide with water.
